(19) United States
(12) Patent Application Publication
(10) Pub. No.: US 2010/0048723 A1
(43) Pub. Date: Feb. 25, 2010

(54) Compositions and Methods for Preventing or Treating Obesity In Animals

(76) Inventors: Ryan Michael Yamka, Topeka, KS (US); Kim Gene Friesen, Carthage, IN (US)

Correspondence Address: COLGATE-PALMOLIVE COMPANY, 909 RIVER ROAD, PISCATAWAY, NJ, 08855, US

(21) Appl. No.: 12/528,391

(22) PCT Filed: Feb. 23, 2007
(86) PCT No.: PCT/US07/62714

§ 371 Date: Aug. 24, 2009

(51) Int. Cl. A61K 31/16; C07C 233/00

(52) U.S. Cl. 514/627; 564/207

(57) ABSTRACT

Compositions useful to prevent or treat obesity in an animal are disclosed. The compositions comprise a capsaicinoid, preferably capsaicin, or an active metabolite thereof, in an amount effect to prevent or treat obesity in an animal. Also disclosed are methods to prevent or treat obesity in an animal comprising administering a composition of the present invention.

### FIELD OF THE INVENTION

The present invention relates to compositions and methods for preventing and/or treating obesity in animals.

### BACKGROUND OF THE INVENTION

It is an unfortunate fact that many animals, particularly companion animals (e.g., dogs and cats) weigh more than their ideal body weight. Having an excess of adipose tissue, these animals may be clinically diagnosed as, e.g., “fat”, “overweight”, or “obese” and, as such, are more likely to suffer from associated physical disorders such as arthritis, heart disease, respiratory disease, diabetes, bladder cancer, hypothyroidism, joint disease and/or pancreatitis.

Attempts to prevent or reduce the amount of excess adipose tissue on companion animals typically include dietary restriction and exercise. Indeed, many “low” or “reduced” calorie foods for overweight companion animals have been developed and are commercially available for the control of calorie intake, prevention of weight gain or for the promotion of weight loss. Despite the availability of such pet food formulations, however, the need remains for the development of additional formulations comprising innovative ingredients and nutrients for preventing or treating obesity in companion animals.

Employing canine cell culture genomic assays, we have identified substances that can modify the expression of various canine genes in vitro. Given these data along with recent findings which suggest that the expression of certain genes may be modified during energy (caloric) restriction in mice (Higami, Y. et al. J. Nutr. 2006 February; 136(2):343-52), we've identified a list of substances that may be useful to prevent or treat obesity in companion animals. Specifically, and as described hereinbelow, in vivo data gathered from intake studies indicate that compositions comprising capsaicin are particularly effective to prevent or treat obesity in animals.

Capsaicin, an alkaloid well known as the source of the “heat” or pungency in chili peppers, has been used in numerous ways, including, for example, as an insect and animal repellant as well as in creams and gels for medical use. In addition, U.S. Pat. No. 5,273,754 discloses methods for decreasing appetite by oral administration of an appetite suppressant composition comprising a “heating carminative substance”, preferably capsaicin, and a “cooling carminative substance”. This reference, however, does not disclose compositions comprising capsaicin without the addition of the cooling carminative (e.g., peppermint, spearmint, menthol) nor does it disclose compositions comprising capsaicin as animal feed.

### SUMMARY OF THE INVENTION

The present invention relates to compositions that are useful to prevent and/or treat obesity in an animal. Said compositions comprise substances that can modify, e.g., up or down regulate, the expression of one or more genes associated with energy restriction in an animal. Thus, in one aspect, the invention relates to a composition comprising a capsaicinoid or an active metabolite thereof, in an amount effective to prevent or treat obesity in an animal. In one embodiment, the capsaicinoid is capsaicin or an active metabolite thereof. In another embodiment, the capsaicinoid is dihydrocapsicin or an active metabolite thereof. In an additional embodiment, the composition may further comprise any one or more of soluble fiber, methionine, eicosapentaenoic acid (EPA) or docosahexanenoic acid (DHA). In one embodiment, the composition is a wet pet food. In another embodiment, the composition is a dry pet food. In one embodiment, the animal to be treated is a canine. In another embodiment, the animal to be treated is a feline.

In another aspect, the invention relates to methods to prevent and/or treat obesity in an animal by administering to said animal a composition comprising a substance which can modify, e.g., up or down regulate, the expression of one or more genes associated with energy restriction in an animal, in an amount effective to prevent or treat obesity in said animal. In one embodiment the composition comprises a capsaicinoid or an active metabolite thereof. In one embodiment, the capsaicinoid is capsaicin or an active metabolite thereof. In another embodiment, the capsaicinoid is dihydrocapsicin or an active metabolite thereof. In an additional embodiment, the composition further comprises any one or more of soluble fiber, methionine, EPA and DHA. In one embodiment, the composition is a wet pet food. In another embodiment, the composition is a dry pet food. In one embodiment, the animal to be treated is a canine. In another embodiment, the animal to be treated is a feline.

In a further aspect, the invention relates to a composition comprising a capsaicinoid, or an active metabolite thereof, in an amount effective to cause the self-regulation of food intake in an animal. In one embodiment, the capsaicinoid is capsaicin or an active metabolite thereof. In another embodiment, the capsaicinoid is dihydrocapsicin or an active metabolite thereof. In an additional embodiment, the composition further comprises any one or more of soluble fiber, methionine, EPA and DHA. In one embodiment, the composition is a wet pet food. In another embodiment, the composition is a dry pet food. In one embodiment, the animal to be treated is a canine. In another embodiment, the animal to be treated is a feline.

In an additional aspect, the invention relates to a method to cause the self-regulation of food intake in an animal comprising administering to said animal a composition comprising a capsaicinoid or an active metabolite thereof, in amount effective to cause said self-regulation of food intake. In one embodiment, the capsaicinoid is capsaicin or an active metabolite thereof. In another embodiment, the capsaicinoid is dihydrocapsicin or an active metabolite thereof. In an additional embodiment, the composition further comprises any one or more of soluble fiber, methionine, EPA and DHA. In one embodiment, the composition is a wet pet food. In another embodiment, the composition is a dry pet food. In one embodiment, the animal to be treated is a canine. In another embodiment, the animal to be treated is a feline.

In a further aspect, the invention relates to the use of a capsaicinoid, or an active metabolite thereof, in the manufacture of a composition for preventing or treating obesity in an animal. In a particular embodiment, the capsaicinoid is capsaicin or an active metabolite thereof. In another embodiment, the capsaicinoid is dihydrocapsicin or an active metabolite thereof. In an additional embodiment, the composition may further comprise any one or more of soluble fiber, methionine, EPA and DHA. In one embodiment, the composition is a wet pet food. In another embodiment, the composition is a dry pet food. In one embodiment, the animal to be treated is a canine. In another embodiment, the animal to be treated is a feline.

In a further aspect, the invention relates to the use of a capsaicinoid, or an active metabolite thereof, in the manufacture of a composition for causing the self-regulation of food intake in an animal. In a particular embodiment, the capsaicinoid is capsaicin or an active metabolite thereof. In another embodiment, the capsaicinoid is dihydrocapsicin or an active metabolite thereof. In an additional embodiment, the composition may further comprise any one or more of soluble fiber, methionine, EPA and DHA. In one embodiment, the composition is a wet pet food. In another embodiment, the composition is a dry pet food. In one embodiment, the animal to be treated is a canine. In another embodiment, the animal to be treated is a feline.

In further aspects, the invention relates to the use of the compositions of the present invention in methods to prevent or treat obesity in an animal and methods to cause the self-regulation of food intake in an animal as described in detail herein.

In a further aspect, the invention relates to a kit which comprises the compositions of the present invention.

Other features and advantages of the present invention will be understood by reference to the detailed description of the examples that follow.

### DETAILED DESCRIPTION OF THE INVENTION

It is contemplated that the invention described herein is not limited to the particular methodology, protocols, and reagents described as these may vary. It is also to be understood that the terminology used herein is for the purpose of describing particular embodiments only, and is not intended to limit the scope of the present invention in any way.

Unless defined otherwise, all technical and scientific terms used herein have the same meanings as commonly understood by one of ordinary skill in the art to which this invention belongs. All patents, patent applications, publications, and other references cited or referred to herein are incorporated by reference for all purposes.

As used herein and in the appended claims, the singular forms “a”, “an”, and “the” include plural reference unless the context clearly dictates otherwise.

As used herein, the terms “overweight”, “fat”, “obese”, “obesity” and like terms refer to a body weight condition of an animal that is more than its' ideal weight. For example, the term “fat” as applied to an animal can mean any animal that is determined to have an excess amount of body adipose tissue or an animal that is prone to developing an excess amount of body adipose tissue using techniques and methods known to veterinary care professionals and others of skill in the art. For example, an animal is considered “fat” if (1) the animal has a Body Mass Index (BMI) of 25 or more (a number considered to include “overweight” and “obese” animals in some methods of characterizing animal conditions), (2) the animal's weight is 15% or more than its “ideal” body weight as defined by veterinary care professionals or as known to one of skill in the art, (3) an animal's percent body fat is 27% or more as determined by DEXA, or (4) an animal has a body condition score (BCS) of more than 3 on a scale from 1 to 5 as determined by one of skill in the art using the method disclosed in “Small Animal Clinical Nutrition”, 4^{th} Edition, in Chapter 13 (ISBN 0-945837-05-4) or its equivalent using other BCS methods. In some cases, animals that are 20% or more over ideal body weight are considered obese. As used herein in relation to the methods and compositions of the present invention, the term “obesity” includes any body weight condition that would be deemed by one of skill in the art as being more than ideal for the animal.

The present invention relates to any animal, preferably a mammal, more preferably a companion animal. The term “companion animal” refers to any animal that lives in close association with humans and includes, but is not limited to, canines and felines of any breed. For example, it is contemplated herein that this term may also encompass any animal whose diet may be controlled by humans and which may benefit from feeding the formulations disclosed herein. These animals may include, for example, domesticated farm animals (e.g. cattle, horses, swine, etc.) as well as undomesticated animals held in captivity, e.g. in zoological parks and the like.

As used herein, “an amount effective to prevent or treat obesity”, “an effective amount”, and like terms refer to that amount of a compound, material or composition as described herein that may be effective to achieve a particular biological result. Such results may include, but are not limited to, the treatment and/or prevention of obesity. Such effective activity may be achieved, for example, by administration of compositions of the present invention to an animal in need thereof.

As used herein, any named substance includes not only the substance but also any chemical equivalent, active metabolite or salt thereof, as the case may be. For example, the term “vitamin” as provided herein is meant to include any and all chemical forms or equivalents of these compounds as may be appropriate for formulation in the compositions of the present invention as readily discerned by one of skill in the art.

As used herein “capsaicinoid” refers to the chemical compounds responsible for the sensation of “heat” or pungency typically associated with the fruits of the Capsicum genus of plants, e.g., chili peppers. This class of compounds includes, capsaicin (i.e., 8-methyl-N-vanillyl-6-nonenamide), dihydrocapsaicin, nordihydrocapsaicin, homocapsaicin and homodihydrocapsaicin, the structure and chemical formulas of which are familiar to one of skill in the art. As used herein, the term includes naturally occurring capsaicinoids (e.g., isolated from chili peppers or paprika) as well as any and all synthetic forms, and any salts or metabolites thereof. The nature of the capsaicinoid may be as a powder, oil, resin or other form suitable for formulating with the compositions disclosed herein. Commercially available forms include, e.g., the dried fruit of Capsicum frutescens L. or Capsicum annuum L., e.g., commercially available as “oleoresin capsicum, African type” (referred to simply as “capsicum” in the examples below) (KALSEC, Kalamazoo, Mich.). As contemplated herein, the compositions of the present invention may comprise one or more capsaicinoids.

As used herein, “soluble fiber” refers to dietary fiber that attracts water during digestion and slows the rate of nutrient absorption and is typically found in, e.g. oat bran, seeds, beans, and certain fruits and vegetables such as beet pulp, guar gum, chicory root, psyllium, pectin, blueberry, cranberry, squash, apples, oats, beans, citrus, barley, or peas. As used herein, the term encompasses any source of soluble fiber suitable for the compositions disclosed herein as would be evident to one of skill in the art.

As used herein, the term “modify the expression of one or more genes associated with energy restriction in an animal” refers to the up or down regulation of mRNA levels of a gene such that levels of proteins encoded by the genes may be correspondingly modified, resulting in a beneficial effect on an animal's weight. Without being limited to any particular theory, changes in expression may be due to modification in the stability of mRNA transcripts, and/or modification in the actual level of transcription.

“Genes associated with energy restriction in an animal” refers to those genes that are modified in an animal subjected to energy, or caloric, restriction. Without being limited to any particular theory, examples of such genes include those discussed in Higami, Y. et al. J. Nutr. 2006 February; 136(2):343-52, and are suspected to include genes associated with inflammation, the cytoskeleton, the extracellular matrix and angiogenesis. Table 1A disclosed herein below lists some corresponding canine genes the expression of which is modified in vitro in the presence of various compounds; as disclosed herein, capsaicin can modify several of these genes in vitro (indicated by “*”).

TABLE 1A
Genes associated with energy restriction in mice
(Higami et al.) also modified in canine cells by
exposure to compounds in vitro

Complements, their regulators and their receptors
Complement C1q A chain
Complement component 4 *
CD59 antigen
Histidine-rich glycoprotein
Interferon regulatory transcription factors and
interferon-induced proteins
Fragilis/homolog to rat interferon-inducible
protein*
Homolog to Homo sapiens interferon-induced 35-
kDa protein*
Acute phase proteins
Haptoglobin
Serum amyloid A 3 protein
MHC and its regulators
Regulatory factor X-associated ankyrin-containing
protein
Protein degradation
Cathepsin S
Cathepsin Z
Cell type-specific
Lysosomal-associated multitransmembrane protein
CD81 antigen
Natural resistance-associated macrophage protein 1
^{α} Cytochrome b558/cytochrome b-245 *
CD68 antigen
EGF-like module containing, mucin-like hormone
receptor 1; EMR1
TYRO protein tyrosine kinase binding protein;
TYROBP
Fyn proto-oncogene
Inflammation, others
Annexin A1
MD-1/lymphocyte antigen 86
Tubulin
^{α} Tubulin, 1 *
^{α} Tubulin, 6
Tubulin, β 2
Tubulin, β 3
Tubulin, β 5
Other cytoskeleton
^{γ} Actin, cytoplasmic
Spectrin, β, brain
Actin-modulating
^{ε} Chaperonin containing Tcp1 subunit
Actin-related protein 2/3 complex, subunit 2, 34 kDa
L-Plastin/lymphocyte cytosolic protein 1*
Septin 9
Thymosin, β 10
Collagen
^{α} Procollagen, type I, 1
^{α} Procollagen, type V, 1
Collagen modulating
Lysyl oxidase; Lox
Other extracellular matrix
Nidogen 2; NID2/osteonidogen
Fibulin 2
Cell adhesion
^{δ} Sarcoglycan,
Inhibitor of DNA binding 1
^{α} Hypoxia inducible factor 1, subunit
Extracellular matrix
Thrombospondin 1
Protein turnover
Mesoderm specific transcript
Serine proteinase inhibitor, clade B, member 6

The present invention is based on the surprising discovery that, in vitro, certain substances can modify (e.g., up or down regulate) the expression of many canine genes that are also modified during energy restriction in mice, and in particular, in vivo data disclosed herein indicate that compositions comprising a capsaicinoid, e.g., capsaicin, may be useful to treat or prevent obesity in an animal. Specifically, but without being limited to any particular theories or modes of action of the invention, data indicate that the addition of capsaicin to a food composition can result in a self-reduction in food intake by a companion animal. Thus, in one particular aspect, the invention relates to compositions useful to prevent and/or treat obesity in an animal, the compositions comprising a capsaicinoid, preferably capsaicin, in an amount effective to treat or prevent obesity in an animal. In another aspect, the invention relates to methods to prevent and/or treat obesity in an animal comprising administering to the animal a composition comprising a capsaicinoid, preferably capsaicin, in an amount effective to treat or prevent obesity in the animal.

As used herein, “the self-regulation of food intake in an animal” refers to the tendency of an animal to ingest a quantity of food until saited, and not to ingest further amounts of food beyond this amount, which can lead to obesity. For example, such tendency may be characterized by the animal (e.g., a canine) walking away from a food bowl still containing food, the animal having reached the feeling of satiety upon ingestion of a lesser amount of a composition of the present invention than when fed other food compositions. With regard to felines, which typically leave food in their bowls only to return and eat again at various times thereafter, it is contemplated herein that the compositions of the present invention may cause an overall decrease in the total amount of food actually ingested by the feline upon each visit to their food bowl.

As used herein, an “ingredient” refers to any component of a composition. “Nutrient” refers to a substance that provides nourishment. In some cases, an ingredient may comprise more than one “nutrient”, for example, a composition may comprise fish oil as an ingredient, the oil itself comprising important nutrients such as EPA and DHA. The distinction in these terms is familiar to one of skill in the art.

As contemplated herein, the compositions of the present invention are meant to encompass nutritionally complete and balanced animal feed compositions that additionally comprise a capsaicinoid, preferably capsaicin. In additional aspects, the compositions of the present invention may further comprise any one or more of soluble fiber, methionine, EPA and DHA.

Nutritionally complete and balanced pet food compositions are familiar to one of skill in the art. For example, substances such as nutrients and ingredients suitable for nutritionally complete and balanced animal feed compositions, and recommended amounts thereof, may be found, for example, in the Official Publication of the Association of American Feed Control Officials, Inc. (AAFCO), Atlanta, Ga. 2005, or the National Research Council's Nutrient Requirements of Dogs and Cats, The National Academy Press, Washington, D.C., 2006.

For example, a nutritionally complete and balanced dog food composition of the present invention may comprise:
about 0 to about 90%, preferably about 5% to about 45%, by weight of carbohydrates;
about 5% to about 70%, preferably about 10% to about 60%, by weight of protein;
about 2% to about 50%, preferably about 5% to about 40%, by weight of fat;
about 0.1% to about 20%, preferably about 1% to about 1 1%, by weight of total dietary fiber;
about 0 to about 15%, preferably about 2% to about 8%, by weight of vitamins and minerals, antioxidants, and other nutrients which support the nutritional needs of the animal; and
about 25 ppm to about 200 ppm, preferably about 50 ppm to about 125 ppm, capsaicin.

For example, a nutritionally complete and balanced cat food composition of the present invention may comprise:
about 0 to about 90%, preferably about 5% to about 45%, by weight of carbohydrates;
about 5% to about 70%, preferably about 20% to about 60%, by weight of protein;
about 2% to about 50%, preferably about 5% to about 40%, by weight of fat;
about 0.1% to about 20%, preferably about 1% to about 11%, by weight of total dietary fiber;
about 0 to about 15%, preferably about 2% to about 8%, by weight of vitamins and minerals, antioxidants and other nutrients which support the nutritional needs of the animal; and
about 25 ppm to about 200 ppm, preferably about 50 ppm to about 125 ppm, capsaicin.

Among other things, as discussed above, the compositions of the present invention may additionally comprise any one or more of soluble fiber, methionine, EPA and DHA. Suitable amounts of each may be easily determined by one of skill in the art without undue experimentation, for example, by conducting bioavailability, heat stability and/or efficacy studies according to conventional methods used for formulating pet food compositions. For example, amounts may range from about 0.3% to about 7% soluble fiber, from about 0.50% to about 1.50% methionine, from about 0.10% to about 0.60% EPA, and from about 0.10% to about 0.50% DHA.

Compositions of the present invention can additionally contain additives typically known in the art, for example stabilizers and processing aids, in amounts which do not impair the purpose and effect provided by the invention. Stabilizers can be added to increase the shelf life of the composition by supplementing or reinforcing the effect of the physical methods used to increase the shelf life. Examples of stabilizers are preservatives, antioxidants, synergists and sequestrants, packaging gases, emulsifiers, thickeners, gelling agents and humectants. Examples of emulsifiers and thickening agents include, but are not limited to, gelatin, cellulose ethers, starch, starch esters, starch ethers and modified starches. Other additives known in the art may be used for nutritional and palatability purposes, suitable amounts of which may be easily determined by a person having ordinary skill in the art. For example, up to about 5% by weight of such additives may be used. Additives may also include, but are not limited to, iron oxide, sodium chloride, potassium citrate, potassium chloride and other edible salts, flavorings, vitamins, minerals and coloring.

While foods of any consistency or moisture content are contemplated, preferably the compositions of the present invention may be, for example, a wet or dry animal food composition. “Wet” food refers to food that is sold in cans or foil bags and has a moisture content of about 70 to about a 90%. “Dry” food refers to compositions with about 5 to about 15% moisture content and is often manufactured in the form of small bits or kibbles. Also contemplated herein are compositions that may comprise components of various consistency as well as components that may include more than one consistency, for example, soft, chewy meat-like particles as well as kibble having an outer cereal component and an inner cream component as described in, e.g., U.S. Pat. No. 6,517,877.

It is contemplated herein that conventional manufacturing methods familiar to one of skill in the art may be utilized to manufacture the compositions of the present invention. The capsaicinoid may be added at any time in the manufacturing processes, however, particular care should be taken to avoid any possible loss of activity or “heat” (e.g., as measured in Scoville units) during manufacture by heat treatment as well as any unnecessary exposure to the capsaicinoid during the manufacturing process given the volatile nature of these compounds.

As contemplated herein, the compositions of the present invention may comprise capsaicinoids in amounts of active ingredient from about 25 ppm to about 200 ppm, preferably from about 50 ppm, more preferably from about 125 ppm. Significantly, the amount of capsaicinoid should not be so great such that it causes an adverse, unpleasant or harmful physical effect to the animal upon ingestion. As disclosed in the examples provided hereinbelow, the compositions of the present invention comprise amounts of capsaicinoids that are effective to prevent or treat obesity in an animal but neither deter the animal from ingesting the food nor cause the animal any physically harmful effects.

As discussed above, another particular aspect of the invention relates to methods to treat or prevent obesity in an animal by administering to the animal an effective amount of a composition comprising one or more capsaicinoids. Preferably, the capsaicinoid is capsaicin. The compositions and methods of the present invention may be part of an overall weight loss program, for example, the compositions and methods may be administered in conjunction with regular exercise and restricted access to treats, table scraps or other pet snacks. In addition, as the compositions of the present invention cause the animal to self-regulate the amount of food ingested, it is contemplated that the compositions disclosed herein may also be particularly useful for pet owners who may not be particularly mindful of the body condition or health status of their animal, e.g., pet owners who may have a tendency to overfeed their pet and/or who are less inclined to spend time exercising their animals.

In addition, the invention relates to the use of a capsaicinoid, or an active metabolite thereof, in the manufacture of a composition for preventing or treating obesity in an animal, as well as in the manufacture of a composition for causing the self-regulation of food intake in an animal. With regard to either use, in particular embodiments, the capsaicinoid is capsaicin or an active metabolite thereof. In another embodiment, the capsaicinoid is dihydrocapsicin or an active metabolite thereof. In an additional embodiment, the composition may further comprise any one or more of soluble fiber, methionine, EPA and DHA. In one embodiment, the composition is a wet pet food. In another embodiment, the composition is a dry pet food. In one embodiment, the animal to be treated is a canine. In another embodiment, the animal to be treated is a feline.

The present invention also includes the use of any of the compositions disclosed herein in methods to prevent or treat obesity in an animal comprising administering to said animal a composition of the present invention in an amount effective to prevent or treat obesity in said animal. Similarly, the invention also includes the use of any of the compositions disclosed herein in methods to cause the self-regulation of food intake in an animal comprising administering to said animal said composition in an amount effective to cause the self-regulation of food intake in the animal.

It is also contemplated herein that the present invention includes kits comprising the compositions of the present invention. In addition to containing a composition of the present invention, the kit may further include, e.g., information regarding the problem of obesity in companion animals and instructions for a pet owner who is interested in improving the overall body condition of their companion animal, including feeding the compositions of the present invention. The kit may further comprise instructions and means for a pet owner to monitor the body condition of their companion animal, materials and instructions for recording the animal's weight loss, such as, but not limited to, conventional notebooks or logs for recording an animal's weight loss as well as electronic or other means, such as computer software, suitable for compiling such data electronically. The kit may further comprise exercise equipment suitable for both pet and pet owner such as balls, flying disks, FRISBEES, leashes with pedometers or like devices, or other items as might be useful as part of a total weight loss kit. In addition, the kit may include low calorie snacks or treats for a companion animal being fed a composition of the present invention. The kit may be in any form suitable for commercial or retail sale in, e.g., grocery, pet or discount stores as well as in a veterinarian's office. Conventional methods for manufacturing and packaging such a kit as described herein may be used and would be familiar to one of skill in the art.

The following examples further illustrate the present invention and are not intended to limit the invention.

### Examples

### Example 1

### Generation of a Database of Substances that can Modify the Expression of Canine Genes In Vitro

We have compiled a database of substances known to modify the expression of various canine genes in canine cells in vitro according to the following methods provided below and previously described (see, e.g., U.S. 2006-0200320 Al).

Test substances are assayed in two concentrations using the following canine cell lines: CCL34 (kidney), CRL1430 (thymus), CCL183 (bone) (all obtained from the American Tissue Culture Collection) and CTAC (thyroid) (see, Measurement of NK Activity in Effector Cells Purified from Canine Peripheral Lymphocytes, Veterinary Immunology and Immunopathology, 35 (1993) 239-25 1). Briefly, cells are cultured in OptiCell chambers according to conventional methods and the next day treated with test substance. The solvent used at the higher of the two concentrations is used as a control. The following day the cells are harvested for genomic assay.

RNA is isolated from the canine cultures using commercially available methods, e.g., an RNAeasy Mini Kit (Qiagen, Valencia, Calif.) according to the manufacturers instructions and conventional methods. The quality of the isolated RNA is analyzed using a RNA 6000 Nano Assay and an Agilent 2100 Bioanalyzer. The procedure is performed according to the manufacturer's instructions provided in the Reagent Kit Guide (see Edition November 2003, by Agilent Technologies) with the following modifications: during gel preparation, rather than separating the filtered gel into aliquots of 65 uL each, the stock filtered gel is kept in the original microcentrifuge tube and the 65 uL is aliquoted as needed; while loading the RNA 6000 Nano Marker, 1 uL of RNase-free water (instead of RNA 6000 Nano Marker) is added to each sample well that will not contain sample. When loading the RNA 6000 Ladder and samples, heat denature the samples and RNA 6000 Ladder for an additional 30 seconds (total of 2.5 minutes) at 71° C.; with regard to “Starting the Chip Run”, choose the “Eukaryote Total RNA Nano” option from the assay menu.

Gene expression is measured for the treatment cell lines and controls using Affymetrix canine gene chips Canine-1 and Canine-2 according to the manufacturer's instructions. Gene expression data is determined to be either “up” or “down”-regulated for any given treatment. The decision on whether a gene is “up” or “down” is based on the fold change, which is calculated as treatment intensity/control intensity for each individual probe. The fold change is considered down-regulated if its value is < 1/1.5 (for across all 4 cell lines analysis) or <½ (for within cell lines analysis) and is up-regulated if it is >1.5 (for across all 4 cell lines analysis) or >2 (for within cell lines analysis). Also, a probe is considered significant for further scrutiny if it is called as “present” in only one of the conditions being compared (treatment or control) and is “absent” or “marginal” in the other and the fold change is significant according to the software used. Probes that appear to be regulated in opposite directions in the two treatments are excluded from further analysis. The raw data is analyzed using GeneSpring version 7.0 (GS) software (Agilent Corporation) and validated using the R-Bioconductor (RB) freeware. Both software packages are used to compute probe intensities from the CEL files generated by the Affymetrix Instrument. The Present/Absent/Marginal calls per probe and P-values are computed using the R-Bioconductor and GeneSpring software separately.

Two schemes are used for data analysis. First: “across cell lines” and “within individual cell lines.” In the first scheme, genes are selected for scoring provided they are found to be significant and common across all cell-lines. The “across cell lines” yields the highest confidence data with minimum noise and may provide the best possible clues as to which genes are affected by individual ingredients. In the second scheme, only those genes that show a significant fold change in the two treatments according to both software packages within an individual cell lines are scored.

Table 1A provided hereinabove is a list of canine genes the expression of which is modified in vitro in the presence of various compounds. Specifically, those genes that are modified by capsaicin are identified by an asterix (*).

### Example 2

### In Vivo Canine Intake Study

Based on the in vitro data in Example 1, an in vivo canine intake study testing a food comprising capsaicin is performed as provided below:

Ten overweight dogs with body condition scores greater than “4” out of a possible “5” are identified according to conventional methods for use in the study. The ten dogs are fed a control food (Tables 1 and 2) for a period of two weeks. Food intake is recorded daily and body weights are recorded weekly. At the end of the two-week feeding phase, blood samples are drawn according to conventional methods and analyzed for chemistry screens (see Table 3). Following the two-week feeding of the control food, dogs are then fed the new treatment food containing “capsicum” (Kalsec, Kalamazoo, Mich.) (prepared according to conventional methods) for two weeks. Food intake is recorded daily and body weights are recorded weekly. At the end of this two week feeding, blood samples are drawn and analyzed for chemistry screens. (Table 3)

Results indicate that dogs consuming foods containing capsicum have a reduction in food intake; as a result, the dogs fed the diet containing capsicum lost 0.13 kg. Interestingly, these dogs also have a reduction in blood glucose levels, serum urea nitrogen, alanine amino transferase and alkaline phosphatase (Tables 3-5).

TABLE 1
Ingredient content of treatment food containing capsicum

| | Control | Treatment |
|---|---|---|
| Ingredient, % | Food | Food |
| Rice | 40.37 | 40.37 |
| Poultry By-Product Meal | 25.00 | 25.00 |
| Corn Starch | 20.00 | 19.00 |
| Choice White Grease | 8.00 | 8.00 |
| Cellulose | 2.50 | 2.50 |
| Soybean Oil | 1.00 | 1.00 |
| Capsicum | — | 1.00 |
| Pal Enhancer | 1.00 | 1.00 |
| Potassium Chloride | 0.80 | 0.80 |
| Dicalcium Phosphate | 0.65 | 0.65 |
| Salt Iodized | 0.25 | 0.25 |
| Calcium Carbonate | 0.15 | 0.15 |
| Choline Chloride | 0.14 | 0.14 |
| Vitamin Mix | 0.10 | 0.10 |
| Mineral Mix | 0.04 | 0.04 |
| Total | 100.00 | 100.00 |

TABLE 2
Nutrient content of treatment food containing capsicum

| | Control | Treatment |
|---|---|---|
| Dry Matter Analyticals | Food | Food |
| Ash, % | 4.6 | 4.5 |
| Calcium, % | 0.8 | 0.8 |
| Capsaicin, ppm | 0.0 | 21.3 |
| Chloride-Soluble, % | 0.8 | 0.8 |
| Cystine, % | 0.3 | 0.3 |
| Dihydrocapsaicin, ppm | 0.0 | 32.0 |
| Fat, crude, by Acid Hydrolysis, % | 14.8 | 16.1 |
| Fiber, Crude in Food, % | 2.1 | 1.9 |
| Magnesium, % | 0.1 | 0.1 |
| Methionine, % | 0.5 | 0.5 |
| Phosphorus, % | 0.7 | 0.7 |
| Potassium, % | 0.8 | 0.8 |
| Protein (via Kjeldahl mtd), % | 22.0 | 22.7 |
| Sodium, % | 0.3 | 0.3 |
| Sulfur, % | 0.3 | 0.3 |

TABLE 3
Chemistry screen data of dogs fed the control food
for two weeks and the treatment food for two weeks

| | | | Control |
|---|---|---|---|
| | | | Food vs. |
| | | | Treatment |
| | | | Food |
| | | | (p values) |
| | | Treatment | (NS = not |
| Analyte | Control Food | Food | significant) |
| Serum/plasma glucose (mg/dL) | 91.6 | 76.5 | <0.01 |
| Serum urea nitrogen (mg/dL) | 12.73 | 9.43 | <0.01 |
| Serum creatinine (mg/dL) | 0.6 | 0.6 | 1.000 |
| BUN-Creatinine ratio | 20.92 | 15.62 | <0.01 |
| Serum sodium, (mmol/L) | 148.5 | 148.7 | NS |
| Serum potassium, (mmol/L) | 4.39 | 4.45 | NS |
| Serum chloride, (mmol/L) | 110.9 | 110.6 | NS |
| Serum total calcium (mg/dL) | 11.01 | 11.08 | NS |
| Serum phosphorous (mg/dL) | 4.23 | 4.08 | NS |
| Serum total protein (g/dL) | 6.16 | 6.24 | NS |
| Serum albumin (g/dL) | 3.58 | 3.67 | NS |
| Serum globulin, (g/dL) | 2.58 | 2.57 | NS |
| Serum albumin/globulin ratio | 1.42 | 1.46 | NS |
| Serum alanine aminotransferase | 66.1 | 46.8 | 0.01 |
| (U/L) | | | |
| Serum alkaline phosphatase | 573.9 | 472.6 | 0.09 |
| (U/L) | | | |
| Serum total bilirubin (mg/dL) | 0.2 | 0.19 | NS |
| Serum triglycerides (mg/dL) | 78.6 | 92.4 | NS |
| Serum cholesterol (mg/dL) | 213.2 | 206.6 | NS |
| Serum total magnesium (mg/dL) | 2.44 | 2.47 | NS |
| Serum Sodium/Potassium Ratio | 34.1 | 33.6 | NS |

TABLE 4
Body weight data of dogs fed the control food for two
weeks and the treatment food for two weeks.

| | Control | Treatment |
|---|---|---|
| | Food | Food |
| Average Weight at Beginning, kg | 16.63 | 17.37 |
| Average Weight at Week 1, kg | 17.63 | 17.35 |
| Average Weight at Week 2, kg | 17.37 | 17.24 |

TABLE 5
Intake data of dogs fed the control food for two weeks and
the treatment food for two weeks.

| | Control | Treatment |
|---|---|---|
| | Food | Food |
| Average Intake at Beginning, g | 349.4 | 257.3 |
| Average Intake at Week 1, g | 369.8 | 264.6 |
| Average Intake at Week 2, g | 329.0 | 250.1 |

1. A composition comprising a capsaicinoid, or an active metabolite thereof, in an amount effective to prevent or treat obesity in an animal.

2. The composition of claim 1 wherein the composition can modify the expression of one or more genes associated with energy restriction in an animal.

3. The composition of claim 1 wherein said capsaicinoid is capsaicin or an active metabolite thereof.

4. The composition of claim 1 wherein said capsaicinoid is dihydrocapsaicin or an active metabolite thereof.

5. The composition of claim 1 wherein said composition additionally comprises a substance selected from the group consisting of soluble fiber, methionine, EPA and DHA.

6. The composition of claim 1 wherein said composition is a wet pet food.

7. The composition of claim 1 wherein said composition is a dry pet food.

8. The composition of claim 7 wherein the animal is a canine.

9. The composition of claim 7 wherein said animal is a feline.

10. A method to prevent or treat obesity in an animal comprising administering to said animal a composition comprising a capsaicinoid or an active metabolite thereof, in an amount effective to prevent or treat obesity in said animal.

11. The method of claim 10 wherein said composition can modify the expression of one or more genes associated with energy restriction in an animal.

12. The method of claim 10 wherein said capsaicinoid is capsaicin or an active metabolite thereof.

13. The method of claim 10 wherein said capsaicinoid is dihydrocapsicin or an active metabolite thereof.

14. The method of claim 10 wherein said composition additionally comprises a substance selected from the group consisting of soluble fiber, methionine, EPA and DHA.

15. The method of claim 10 wherein said composition is a wet pet food.

16. The method of claim 10 wherein said composition is a dry pet food.

17. The method of claim 10 wherein said animal is a canine.

18. The method of claim 10 wherein said animal is a feline.

19. The use of a capsaicinoid, or an active metabolite thereof, in the manufacture of a composition for preventing or treating obesity in an animal.

20. The use of a capsaicinoid, or an active metabolite thereof, in the manufacture of a composition for causing the self-regulation of food intake in an animal.

21. The use of claim 19 or claim 20 wherein the capsaicinoid is capsaicin or an active metabolite thereof.

22. The use of claims 19-20 wherein the capsaicinoid is dihydrocapsaicin or an active metabolite thereof.

23. A method to cause the self-regulation of food intake in an animal comprising administering to said animal a composition comprising a capsaicinoid or an active metabolite thereof, in amount effective to cause said self-regulation of food intake.

24. A composition comprising a capsaicinoid, or an active metabolite thereof, in an amount effective to cause self-regulation of food intake in an animal.

25. The use of a composition of any of the preceding claims in a method to cause the self-regulation of food intake in an animal comprising administering to said animal said composition, in an amount effective to cause said self-regulation of food intake.

26. The use of a composition of any of the preceding claims in a method to prevent or treat obesity in an animal comprising administering to said animal said composition in an amount effective to prevent or treat obesity in said animal.

27. A kit comprising a composition of any of the preceding claims.
